(19) United States
(12) Patent Application Publication
(10) Pub. No.: US 2023/0372703 A1
(43) Pub. Date: Nov. 23, 2023

(54) INGESTIBLE ELECTRONIC DEVICE

(71) Applicant: Massachusetts Institute of Technology, Cambridge, MA (US)

(72) Inventors: Carlo Giovanni Traverso, Newton, MA (US); Khalil Ramadi, Brookline, MA (US); James McRae, Cambridge, MA (US)

(73) Assignee: Massachusetts Institute of Technology, Cambridge, MA (US)

(21) Appl. No.: 18/031,256

(22) PCT Filed: Oct. 14, 2021
(86) PCT No.: PCT/US2021/055095

§ 371 Date: Apr. 11, 2023

Related U.S. Application Data
(60) Provisional application No. 63/092,016, filed on Oct. 15, 2020.

(51) Int. Cl. A61N 1/05; A61N 1/36; A61N 1/375

(52) U.S. Cl. CPC A61N 1/0509; A61N 1/36007; A61N 1/3756

(57) ABSTRACT

Embodiments disclosed herein relate to an ingestible electronic device configured to provide short term electrical stimulation to a stomach of a subject. The device may include a non-expandable housing and two or more electrodes disposed on the non-expandable housing to apply the short term electrical stimulation to the stomach of the subject. In some instances, the short term electrical stimulation may be on the order of 1 hour or less. In some instances, the ingestible electronic device may utilize the natural gastrointestinal processes of the subject to retain the ingestible electronic device for an appropriate time period within the stomach. For example, the subject may first ingest the ingestible electronic device and the device may travel into the stomach of the subject. The device may then apply electrical stimulation to the stomach for an appropriate time period prior to passing out of the stomach via a pyloric orifice of the stomach.

### CROSS REFERENCE TO RELATED APPLICATIONS

This application claims the benefit under 35 U.S.C. § 119(e) of U.S. Provisional Application Ser. No. 63/092,016, titled “Ingestible Electronic Device,” filed on Oct. 15, 2020, which is incorporated herein by reference in its entirety.

### FIELD

Disclosed embodiments relate to ingestible electronic devices, for example, ingestible electronic devices for stimulating the release of certain hormones within the human body.

### BACKGROUND

Use of hormonal therapies remains limited due to an inability to administer them orally. Specifically, ingested hormones may be broken down in the gastrointestinal tract, prior to uptake. Correspondingly, hormones are typically injected. This significantly reduces the adherence to hormonal therapies and limits subject compliance. Electroceutical therapies is an emerging approach that uses electrical stimulation of various tissues to affect the metabolism of a subject.

### BRIEF SUMMARY

In one embodiment, an ingestible electronic device includes a non-expandable housing, and the non-expandable housing is configured to pass through a pyloric orifice of the subject without being actively retained in a stomach of the subject. The device also includes at least two electrodes disposed on an exterior surface of the non-expandable housing. The at least two electrodes are configured to apply electrical stimulation to a tissue proximate to the at least two electrodes when the ingestible device is disposed in the stomach of the subject, and the ingestible device is configured to apply the electrical stimulation to the tissue for a time period less than or equal to 1 hour.

In one embodiment, a method of applying electrical stimulation to tissue within a stomach of a subject includes: positioning an ingestible electronic device in the stomach of the subject without actively retaining the ingestible electronic device in the stomach; and applying electrical stimulation to a tissue of the stomach of the subject proximate to the ingestible electronic device for a time period less than or equal to 1 hour.

In one embodiment, an ingestible electronic device includes a non-expandable housing, and the non-expandable housing is configured to pass through a pyloric orifice of the subject without being actively retained in a stomach of the subject. The device also includes at least two electrodes disposed on an exterior surface of the non-expandable housing. The at least two electrodes are configured to apply electrical stimulation to a tissue proximate to the at least two electrodes when the ingestible device is disposed in a stomach of a subject, and the at least two electrodes are configured to contact tissue disposed on multiple sides of the non-expandable housing.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A is a front view of an ingestible electronic device according to one illustrative embodiment;

FIG. 1B is a cross sectional view of the ingestible electronic device of FIG. 1A along line 1B-1B according to one illustrative embodiment;

FIG. 2 shows an illustrative embodiment of an ingestible electronic device traveling through the gastrointestinal tract of an exemplary subject;

FIG. 3A is a chart showing the operation time of an ingestible electronic device according to one exemplary embodiment;

FIG. 3B is a graph showing the change in Ghrelin plasma concentration in a subject using the stimulation timing shown in FIG. 3A versus no stimulation; and

FIG. 4 is a graph of the change in Ghrelin plasma concentration in subjects for different stimulation times;

FIG. 5A is a front view of an ingestible electronic device according to one illustrative embodiment;

FIG. 5B is a front view of an ingestible electronic device according to one illustrative embodiment;

FIG. 5C is a front view of an ingestible electronic device according to one illustrative embodiment;

FIG. 5D is a front view of an ingestible electronic device according to one illustrative embodiment;

FIG. 6A is a front view of an ingestible electronic device according to one illustrative embodiment;

FIG. 6B is a front view of an ingestible electronic device according to one illustrative embodiment;

FIG. 7A is an exemplary groove configuration used to collect data in one illustrative experiment;

FIG. 7B is a first chart showing data collected during the experiment of FIG. 7A;

FIG. 7C is a second chart showing data collected during the experiment of FIG. 7A; and

FIG. 8 illustrates the performance of three embodiments of an ingestible device during one exemplary experiment.

### DETAILED DESCRIPTION

To influence the metabolism of a subject, a clinician may administer a hormonal therapy to the subject, which may influence the body of the subject to release certain hormones. For example, in some instances, a clinician may wish to influence the levels of the hormone Ghrelin within a subject. In some instances, electrical stimulation of the stomach mucosa may have a significant effect on the levels of the hormone Ghrelin present in a subject. Ghrelin is an orexigenic (hunger-inducing) hormone that may be released by the stomach of a subject. Increasing the production of Ghrelin in a subject may have a number of effects, including anti-emetic effects. In conventional applications, a clinician may use an electrical stimulation device that is retained within the stomach to apply long term electrical stimulation to the stomach of the subject to increase the subject's Ghrelin levels (e.g., approximately 20 hours or more). Thus, in conventional applications, an electrical stimulation device may include structures for retaining the electrical stimulation device within the stomach of the subject to prevent the electrical stimulation device from passing through the gastrointestinal (GI) tracts of the subject. Some exemplary structures that are used to retain a device within the stomach of a subject include expandable structures and housing, hooks, adhesives, or other suitable retention mechanisms used to maintain a device within the stomach of a subject for long durations.

In contrast to typical systems, the Inventors have recognized that applying short term electrical stimulation (e.g., approximately 1 hour or less) to a stomach of a subject may produce clinically significant changes in the production of Ghrelin in the subject. In fact, the Inventors observed that short term electrical stimulation therapy may achieve similar levels of Ghrelin in a subject as long term conventional electrical stimulation therapy. Thus, the Inventors recognized the need for an electrical stimulation device capable of applying short term electrical stimulation to the stomach of the subject without being retained in the stomach for long durations.

In view of the above, the Inventors have recognized the advantages of an electrical stimulation device capable of providing minimally invasive short term electrical stimulation to the stomach of a subject. For example, in some instances, an electrical stimulation device may take the form of an ingestible electronic device. Such an ingestible electronic device may include two or more electrodes disposed on an external surface of a housing of the ingestible electronic device. With regards to the current disclosure, the use of electrodes that lie flush with surrounding portions of an external surface may still be considered to be disposed on an external surface of the device. Further, the ingestible electronic device may be constructed to pass through the stomach of the subject without the need for a retaining mechanism. The ingestible electronic device may be sized and shaped such that it may leave the stomach of the subject by passing through a pyloric orifice of the subject. To facilitate passage through the pyloric orifice of the subject, the housing of the ingestible electronic device may be non-expandable in some embodiments. Thus, the ingestible electronic device may use the natural gastrointestinal functions to retain the ingestible electronic device for an appropriate time period, then excrete the ingestible electronic device after the appropriate time period has elapsed.

According to one aspect, an ingestible electronic device may be constructed to allow a subject to ingest the ingestible electronic device (e.g., orally). The ingestible electronic device may then travel through an esophagus of the subject into the stomach of the subject. Once the ingestible electronic device enters the stomach of the subject, the ingestible electronic device may then apply electrical stimulation to a tissue within the stomach of the subject (e.g., the tissue of the stomach proximate to the ingestible electronic device). The ingestible electronic device may continue to apply electrical stimulation to the tissue for an appropriate time period. Subsequently, the ingestible electronic device may exit the stomach via a pyloric orifice of the stomach after residing within the stomach for an appropriate time period. Electrical stimulation may stop either prior the device leaves the stomach or after it is determined that the device is no longer in the stomach using timing, available power, and/or active sensing as elaborated on below. Possible stimulation and passage times of an ingestible electronic device as described herein are elaborated on further below.

According to another aspect, an ingestible electronic device may include a housing and at least two electrodes. The housing may be sized and shaped to permit the device to enter the stomach of a subject through the esophagus and exit the stomach of the subject through a pyloric orifice of the stomach. Moreover, in some embodiments, the housing may be non-expandable to prevent the ingestible electronic device from being retained within the stomach of the subject for an extended period of time. The housing may also include an exterior surface with two or more electrodes disposed on the exterior surface. Thus, the two or more electrodes may be configured to apply electrical stimulation to tissue proximate to the external surface of the ingestible electronic device including the electrodes. When the ingestible electronic device approaches and/or comes into contact with a tissue disposed within the stomach of the subject, the ingestible electronic device may provide electrical stimulation to the tissue for a predetermined time period as described herein.

As used herein, the term proximate may refer to the electrodes of an ingestible electronic device being sufficiently close to the mucosal lining of the stomach to conduct a current to the tissue through the intervening material present in the stomach as well as instances in which the electrodes are in direct contact with the mucosal lining of the stomach. For example, in some embodiments, the electrodes and associated power source of an ingestible electronic device may be configured such that they are capable of applying electrical stimulation to tissue of a subject's stomach that is disposed within about 1 cm, 5 mm, 1 mm, and/or any other appropriate distance from the electrodes of a device.

In some embodiments, an ingestible electronic device may not be self-righting within the stomach of a subject. Accordingly, it may not be possible to ensure that a particular surface of the device is oriented in a vertically downwards direction towards an underlying portion of the stomach of a subject relative to a direction of gravity. For example, in some instances, an ingestible electronic device may include multiple pairs of electrodes. In such instances, the multiple pairs of electrodes may be disposed on different portions of the surface of the housing. For example, the ingestible electronic device may include two or more pairs of electrodes disposed on different portions of the device housing to allow at least one of the pairs of electrodes to come into contact with tissue underlying the device in various orientations. In one such embodiment, first and second pairs of electrodes may be disposed on separate portions of a housing of the device including, for example, portions of the device that may be located opposite from one another (e.g. a top surface and a bottom surface of the device). Alternatively, in other embodiments, one or more pairs of electrodes may extend along different portions of an external surface of the housing such that the one or more pairs of electrodes may contact tissue underlying the device in various orientations. For example, the one or more pairs of electrodes may extend helically along a length of a device on an external surface of the device as described further below. However, electrodes that extend along the external surface of the device may have any appropriate shape and/or may follow any appropriate path along the surface of the device as the disclosure is not limited in this fashion. Thus, in some embodiments, the one or more pairs of electrodes provided on a device may be used to provide electrical stimulation to tissue disposed on multiple sides of the housing.

As noted above, the ingestible electronic devices disclosed herein may not be actively retained in a stomach of a subject. In such an embodiment, an ingestible electronic device may freely pass through the stomach of the subject as the device does not adhere, attach, expand, or otherwise operate in a manner to prevent passage of the ingestible electronic device through a pylorus of the stomach. Accordingly, a passage time for an ingestible electronic device may correspond to a duration for the device to pass through a stomach of a subject without any manipulation of the gastric residence time and may be anywhere between about 5 minutes and 4 hours which corresponds to the normal range of residence times for materials located within the stomach of a subject.

The ingestible electronic device may be configured to provide electrical stimulation to the tissue located within the stomach of a subject in any suitable manner (e.g., a manner configured to influence the subject to generate higher levels of Ghrelin). Further, the various operating parameters of the applied electrical stimulation provided by an ingestible electronic device may be modified in any appropriate manner to provide a desired type of electrical stimulation appropriate for a desired application. For example, the applied electrical stimulation may be modified by adjusting the frequency, voltage magnitude, current magnitude, pulse duration, pulse shape, stimulation duration, stimulation timing or duty cycle which may correspond to a percentage of time stimulation may be applied during operation), and/or any other appropriate operating parameter.

The frequency of electrical pulses applied during electrical stimulation of the stomach tissue of a subject may include frequencies that are greater than or equal to 10 Hz, greater than or equal to 11 Hz, greater than or equal to 12 Hz, greater than or equal to 13 Hz, greater than or equal to 14 Hz, greater than or equal to 15 Hz, greater than or equal to 16 Hz, greater than or equal to 17 Hz, greater than or equal to 18 Hz, greater than or equal to 20 Hz, or any other appropriate frequency. In some instances, the frequency less than or equal to 50 Hz, may be less than or equal to 40 Hz, less than or equal to 30 Hz, less than or equal to 20 Hz, less than or equal to 19 Hz, less than or equal to 18 Hz, less than or equal to 17 Hz, less than or equal to 16 Hz, less than or equal to 15 Hz, less than or equal to 14 Hz, less than or equal to 13 Hz, less than or equal to 12 Hz, or any other appropriate frequency. Of course, combinations of the above ranges are also possible. For example, in some instances, a frequency of the applied electrical pulses may be between or equal to 10 Hz and 50 Hz. Of course, different combinations of the above-noted frequency ranges as well as frequencies both greater than or less than those noted above are also contemplated as the disclosure is not so limited.

In some embodiments, the electrical pulses applied by an ingestible electronic device to the tissue within a stomach of a subject may include a pulse duration corresponding to the time associated with an individual electrical pulse being applied to tissue by the ingestible electronic device. For example, in some instances, a pulse duration of an applied electrical stimulation may maintain be greater than or equal to 0.1 ms, 0.25 ms, 0.3 ms, 0.4 ms, 0.5 ms, 1 ms, 2.5 ms, 5 ms, or any other appropriate pulse duration. In some instances, the applied pulse duration may be less than or equal to 10 ms, 5 ms, 2.5 ms, 1 ms, 0.5 ms, 0.4 ms, 0.3 ms, 0.25 ms, or any other appropriate pulse duration. Of course, combinations of the above ranges are also possible. For example, in some instances, the pulse durations associated with an applied electrical stimulation may be between or equal to 0.1 ms and 10 ms. However different combinations of the foregoing ranges as well as pulse durations both greater than and less than those noted above are also contemplated as the disclosure is not limited in this fashion.

In addition to the above noted parameters, the electrical stimulation applied to a subject's stomach may have any suitable current. For example, in some instances, a current applied to the tissue within a subject's stomach may be greater than or equal to 0.5 mA, greater than or equal to 1 mA, greater than or equal to 2 mA, greater than or equal to 3 mA, greater than or equal to 4 mA, greater than or equal to 5 mA, greater than or equal to 6 mA, greater than or equal to 7 mA, greater than or equal to 8 mA, and/or any other appropriate current. In some instances, the current applied during electrical stimulation may be less than or equal to 10 mA, less than or equal to 9 mA, less than or equal to 8 mA, less than or equal to 7 mA, less than or equal to 6 mA, less than or equal to 5 mA, less than or equal to 4 mA, less than or equal to 3 mA, and/or any other appropriate current. Of course, combinations of the above ranges are also possible. For example, in some instances, the current applied during electrical stimulation may be between or equal to 0.5 mA and 10 mA. Of course, different combinations of the above-noted ranges as well as currents both greater than and less than those noted above are also contemplated as the disclosure is not so limited.

The electrical stimulation applied to the tissue of a subject's stomach may also have any suitable voltage. For example, in some instances the applied voltage may be greater than or equal to 0.5 V, greater than or equal to 1 V, greater than or equal to 1.5 V, greater than or equal to 2 V, greater than or equal to 2.5 V, greater than or equal to 3 V, greater than or equal to 3.5 V, greater than or equal to 4 V, greater than or equal to 4.5 V, and/or any other appropriate voltage. In some instances, the applied voltage may be less than or equal to 5 V, less than or equal to 4.5 V, less than or equal to 4 V, less than or equal to 3.5 V, less than or equal to 3 V, less than or equal to 2.5 V, less than or equal to 2 V, less than or equal to 1.5 V, less than or equal to 1 V, and/or any other appropriate voltage. Combinations of the above ranges are also possible. For example, in some instances, the applied voltage may be between or equal to 0.5 V and 5 V. Of course, other combinations of the foregoing ranges as well as voltages both greater than and less than those noted above are also contemplated as the disclosure is not so limited.

An ingestible electronic device may apply a desired electrical stimulation to the tissue of a subject's stomach for any suitable exposure time. For example, in some instances, an electrical stimulation may be applied to the stomach of the subject for a duration that is greater than or equal to 5 minutes, greater than or equal to 10 minutes, greater than or equal to 20 minutes, greater than or equal to 30 minutes, greater than or equal to 40 minutes, greater than or equal to 50 minutes, greater than or equal to 60 minutes, greater than or equal to 70 minutes, greater than or equal to 80 minutes, greater than or equal to 90 minutes, greater than or equal to 100 minutes, greater than or equal to 110 minutes, or any other appropriate duration. In some instances, the electrical stimulation may be applied to the tissue of the stomach of a subject for a duration that is less than or equal to 120 minutes, less than or equal to 100 minutes, less than or equal to 90 minutes, less than or equal to 80 minutes, less than or equal to 70 minutes, less than or equal to 60 minutes, less than or equal to 50 minutes, less than or equal to 40 minutes, less than or equal to 30 minutes, less than or equal to 20 minutes, or less than or equal to 10 minutes. Combinations of the above ranges are also possible. In some instances, the duration may be between or equal to 5 minutes and 120 minutes, 5 minutes and 60 minutes, and/or any other appropriate combination of the above ranges. Of course, durations both greater than and less than those noted above are also contemplated. In either case, as previously noted, in some embodiments, the duration of the applied electrical stimulation may either be selected or actively controlled to be equal to our less than the expected passage time of an ingestible electronic device as it passes through the stomach of a subject.

As elaborated on in the experiments presented below, the ingestible electronic devices described herein may provide a relatively rapid change in Ghrelin plasma concentration of a subject after the initiation of electrical stimulation. For example, within five minutes of the application of electrical stimulation to the stomach tissue of a subject, the Ghrelin plasma concentration of the subject may change relative to a baseline concentration of the subject by an amount greater than or equal to 70 pg/mL, 80 pg/mL, 90 pg/mL, 100 pg/mL, and/or any other appropriate change in Ghrelin plasma concentration. Correspondingly, the Ghrelin plasma concentration of the subject may change by an amount that is less than or equal to 150 pg/mL, 140 pg/mL, 130 pg/mL, 120 pg/mL, 110 pg/mL, 100 pg/mL, and/or any other appropriate change in concentration within five minutes of the initial stimulation of the stomach tissue. Combinations of the foregoing are contemplated including, for example, a change in Ghrelin plasma concentration of the subject within five minutes of the initial application of electrical stimulation to a stomach tissue of the subject relative to a baseline concentration of the subject that may be measured prior to stimulation may be between or equal to 70 pg/mL and 150 pg/mL. Of course, changes in Ghrelin plasma concentration in a subject both greater than and less than those noted above over the initial five minutes after application of electrical stimulation to the stomach tissue of a subject are also contemplated as the disclosure is not so limited. Additionally, a final change in Ghrelin plasma concentration of a subject may also be greater than the values noted above (e.g. after 60 minutes, 90 minutes, and/or any other appropriate time period after the initial electrical stimulation of the stomach tissue). For instance, a final change in Ghrelin plasma concentration of the subject may be between or equal to about 150 pg/mL and 250 pg/mL though other changes in concentration both greater and less than this may also be used as the disclosure is not so limited.

While specific parameters for an applied electrical stimulation are provided above, it should be noted that any appropriate combination of frequency, pulse duration, current, voltage, electrical stimulation, device retention time, or other appropriate operating parameter may be used to provide a desired amount and/or type of electrical stimulation to the tissue of the subject as the disclosure is not limited to the specific type of electrical stimulation depending on the application. Additionally, the applied electrical stimulation may either be constant over time or may vary over time as the disclosure is not limited in this fashion either.

In some instances, an ingestible electronic device may include a trigger mechanism to begin the application of an electrical stimulation to the tissue of a subject's stomach. According to exemplary embodiments described herein, a trigger of an ingestible electronic device may be configured to actuate the ingestible electronic device in the GI tract of a subject at a predetermined time and/or location in the GI tract (e.g., within the stomach). In some embodiments, the trigger may be a passive component configured to interact with the environment of the GI tract to actuate the ingestible electronic device. For example, in some embodiments the trigger may be a sugar plug, or other dissolvable material, configured to dissolve in the GI tract. The dissolvable plug may have a certain thickness and/or shape that at least partly determines the speed at which the sugar plug dissolves and ultimately actuates the ingestible electronic device by exposing a sensor, electrical conduction path, releasing a physical trigger, or any other appropriate arrangement. In another embodiment, the trigger may be at least partially formed by an enteric coating. For example, in some embodiments, a trigger may include both a sugar plug and an enteric coating, as the present disclosure is not so limited. Other appropriate materials for a dissolvable trigger may include, but are not limited to, sugar alcohols such as disaccharides (e.g. Isomalt), water soluble polymers such as Poly-vinyl alcohol, enteric coatings, time-dependent coatings, enteric and time-dependent coatings, temperature-dependent coatings, light-dependent coatings, pH responsive coatings, a dissolvable capsule, and/or any other appropriate material capable of being dissolved within the GI tract of a subject to either expose the electrodes of an ingestible electronic device or otherwise trigger the device as the disclosure is not limited in this fashion. In other embodiments, a sensor or other active component may be used to trigger operation of an ingestible electronic device to apply a desired electrical stimulation. In one such embodiment, a trigger may include a sensor that detects one or more characteristics of the GI tract. For example, a sensor detecting contact with a GI mucosal lining may be used to actuate the device. In embodiments where a sensor is employed, the trigger may either include a processor that controls the power source and/or a connection between the power source and the electrodes to apply the desired electrical stimulation. Alternatively, the trigger may include an appropriate electrical circuit that switches between an open and closed state to apply the desired electrical stimulation based on the one or more characteristics of the GI tract sensed by the one or more sensors. Of course, it should be understood that any suitable active or passive trigger may be employed for an ingestible electronic device to control actuation and application of electrical stimulation within the stomach of the subject as the present disclosure is not so limited.

The ingestible electronic device may also be sized and shaped to be easily ingestible by the subject and/or pass through the pyloric orifice of the subject. Accordingly, the ingestible electronic device may be appropriately small so that the ingestible electronic device may be easily swallowed and subsequently pass through the GI tract of the subject including the esophagus and pyloric orifice within the stomach of the subject. In some embodiments, an ingestible electronic device may have an overall length, such as a maximum dimension along a longitudinal axis of the device, less than or equal to 40 mm, 30 mm, 20 mm, 10 mm, 5 mm, and/or another appropriate length. Correspondingly, an ingestible electronic device may have an overall length greater than or equal to 3 mm, 5 mm, 10 mm, 20 mm, 25 mm, and/or another appropriate length. Combinations of the above-noted ranges are contemplated, including, but not limited to, overall lengths between 5 mm and 30 mm, 10 mm and 30 mm, 5 mm and 20 mm, as well as 5 mm and 10 mm. In some embodiments, an ingestible electronic device may have a maximum external transverse dimension, such as a diameter, less than or equal to 11 mm, 10 mm, 7 mm, 5 mm, and/or another appropriate diameter. Correspondingly, an ingestible electronic device may have a maximum external transverse dimension greater than or equal to 3 mm, 5 mm, 7 mm, 9 mm, and/or another appropriate diameter. Combinations of the above-noted ranges are contemplated, including, but not limited to, maximum external transverse dimensions between 5 mm and 11 mm, 5 mm and 7 mm, as well as 7 mm and 11 mm. In some embodiments, an ingestible electronic device may have an overall volume less than or equal to 3500 mm³, 3000 mm³, 2500 mm³, 2000 mm³, 1500 mm³, 1000 mm³, 750 mm³, 500 mm³, 250 mm³, 100 mm³, and/or any other appropriate volume. Correspondingly, an ingestible electronic device may have an overall volume greater than or equal to 50 mm³, 100 mm³, 250 mm³, 500 mm³, 750 mm³, 1000 mm³, 1500 mm³, 2000 mm³, 2500 mm³, and/or any other appropriate volume. Combinations of the above-noted ranged are contemplated, including, but not limited, volumes between 1000 mm³ and 3000 mm³, 1500 mm³ and 3000 mm³, 50 mm³ and 500 mm³, 50 mm³ and 100 mm³, as well as 2000 mm³ and 3000 mm³. Of course, any suitable overall length, maximum external transverse dimension, and volume may for an ingestible electronic device may be employed, as the present disclosure is not so limited.

According to exemplary embodiments described herein, the ingestible electronic device may be administered to a subject orally. In other embodiments, the ingestible electronic device may be administered nasally as the present disclosure is not so limited. In addition, the ingestible electronic device may be delivered to the stomach of the subject via a percutaneous gastronomy tube, an endoscope, or any other suitable method of delivery.

In some instances, the ingestible electronic device may include electronic components for managing the operation of the ingestible electronic device. For example, the ingestible electronic device may include a power source, a processor, and one or more sensors. The power source may be electrically coupled to the two or more electrodes of the ingestible electronic device. Thus, the power source may serve to provide electrical power to the two or more electrodes so that the two or more electrodes may provide electrical stimulation to the tissue disposed within the stomach of the subject, as described above. Further, the power source may be sized to fit within the ingestible electronic device. The power source may be a battery, capacitor, super capacitor, or any other suitable power source. Alternatively, the power source may correspond to a wireless power source where a power transmission source may be located outside the body of a subject and the power receiver that wirelessly couples with and draws power from the power transmission source may be included in an ingestible electronic device. Accordingly, it should be understood that the disclosure is not limited to the use of any particular type of power source.

In some embodiments, an ingestible electronic device may also include a processor and associated non-transitory processor readable medium including processor readable instructions stored thereon. When the processor executes the processor readable instructions, the processor may operate the power source, two or more electrodes, and/or any other appropriate component of an ingestible electronic device disclosed herein to perform any of the methods disclosed herein and as elaborated on below.

In some instances, material, such as mucous or other bodily fluids, may become trapped between the electrodes of a device and the underlying tissue. This may decrease the effectiveness of the electrodes in delivering a desired amount of current to the tissue. Accordingly, in some embodiments, the ingestible devices described herein may include features that allow the ingestible device to decrease an impedance between the electrodes and the tissue. For example, in some embodiments, the ingestible device may be configured to wick one or more gastrointestinal fluids from between an exterior surface of the non-expandable housing of the ingestible device and a surface of the underlying tissue when the device is positioned in the stomach. For example, the exterior surface may include one or more grooves, posts, and/or any other suitable structure configured to wick the exterior surface of the ingestible device. Alternatively or in addition, the electrodes positioned on the exterior of the ingestible device may be sized, shaped, and/or oriented such that an impedance between the electrodes and the tissue is decreased. For example, in some embodiments, each electrode may have an overall width, such as a maximum dimension along a lateral axis of the electrode, less than or equal to 405 μm, 400 μm, 395 μm, 390 μm, 385 μm, and/or another appropriate width. Correspondingly, an electrode may have an overall width greater than or equal to 160 μm, 165 μm, 170 μm, 175 μm, 180 μm, and/or another appropriate width. Combinations of the above-noted ranges are contemplated, including, but not limited to, overall widths between 160 μm and 405 μm, 165 μm and 400 μm, 170 μm and 395 μm, as well as 175 μm and 390 μm. The various embodiments described herein may be used with these and/or other dimensions, depending on the application. Correspondingly, in the case of electrodes extending around or along the housing in a direction that is at least partially parallel to another electrode, or portion of the same electrode in the case of a spiral electrode, a pitch between adjacent portions of the one or more electrodes may be in a range similar to the electrode widths noted above.

For embodiments including channels and/or posts formed on the device exterior surface, a maximum transverse dimension (e.g., width or diameter) of the channels and/or posts may be between or equal to 100 μm, 200 μm, 300 μm, 500 μm, 1 mm, and/or any other appropriate dimension. The maximum transverse dimension may also be less than or equal to 2 mm, 1 mm, 500 μm, and/or any other appropriate dimension. Combinations of the foregoing are contemplated including, for example, a maximum transverse dimension that is between or equal to 100 μm and 2 mm. Additionally, a height or depth of the channels and/or posts may be greater than or equal to 100 μm, 200 μm, 300 μm, 500 μm, 1 mm, and/or any other appropriate dimension. The height and/or depth may also be less than or equal to 2 mm, 1 mm, 500 μm, and/or any other appropriate dimension. Combinations of the foregoing are contemplated including, for example, a height and/or depth of the channels and/or posts may be between or equal to 100 μm and 2 mm. A pitch, or other spacing, between adjacent channels and/or posts may correspond to ranges similar to those noted above regarding a pitch between adjacent electrodes.

While particular dimensions related to the use of electrodes, channels, and/or posts are noted above, it should be understood that other ranges of these dimensions, including ranges both greater than and less than those noted above, may also be used as the disclosure is not limited to any particular set of dimensions.

The ingestible electronic devices disclosed herein may also include one or more sensors and/or other circuitry that are configured to send signals related to one or more operating parameters associated with a device and/or an external environment to a processor of the device. The processor may then appropriately control operation of the device based on the received signals. For example, the ingestible electronic device may include a timer configured to determine the total time over which the ingestible electronic device has applied an electrical stimulation to surrounding tissue. The timer may communicate that time to the processor, which in turn, may control operation of the device to continue or discontinue applying the electrical stimulation. For example, if the total stimulation time is less than a preset threshold time (e.g., within the ranges described above), the processor may continue applying electrical stimulation. Conversely, if the timer indicates that the total time is equal to or greater than a preset threshold, the processor may discontinue applying electrical stimulation. In another embodiment, the ingestible electronic device may include a contact sensor configured to sense whether the ingestible electronic device has made contact with tissue located within the stomach of a subject. The processor may then control application of the electrical stimulation to either apply or discontinue application of the electrical stimulation based on whether or not the sensor senses contact with adjacent tissue. In yet another embodiment, a pH sensor may be used to determine whether or not the device is located within a stomach of a subject based on whether or not a sensed pH of the external environment is within a predetermined range of pH values associated with the stomach as opposed to other portions of the gastrointestinal tract. Of course, the ingestible electronic device may include any number of suitable sensors or other appropriate circuitry depending on the application. However, embodiments in which an ingestible electronic device does not include a processor and/or sensors are also contemplated.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

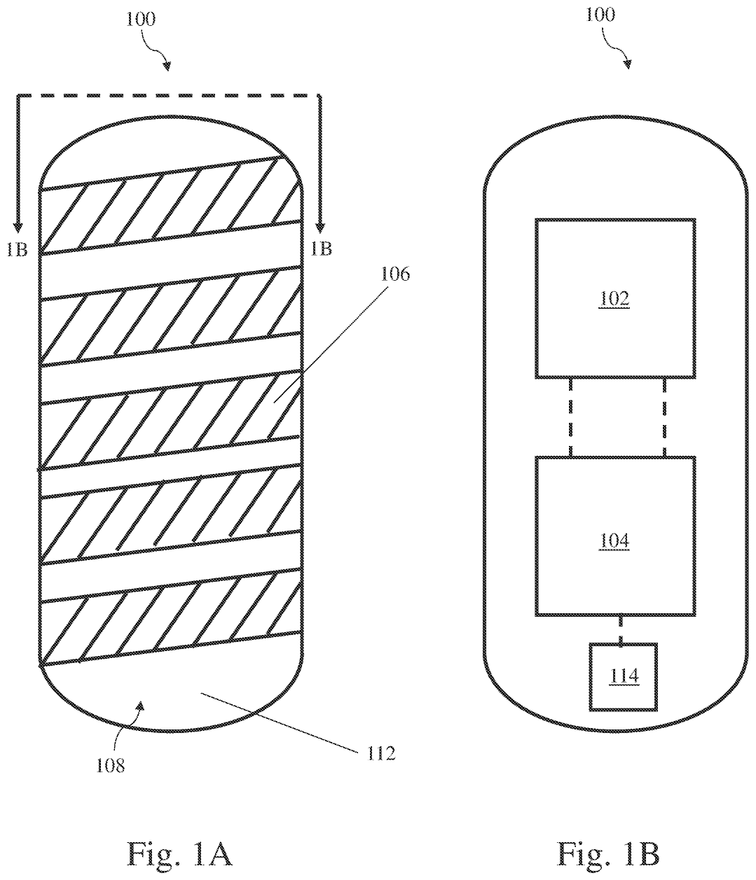

FIG. 1A is a front view of an ingestible electronic device 100 according to an embodiment of the present disclosure. The ingestible electronic device 100 may include a housing 108 and two or more electrodes 106 disposed on an external surface 112 of the housing. The housing 108 may be shaped so as to fit through the esophageal and pyloric orifices of a subject. To avoid long-term retention of the device within a stomach of a subject, housing 108 may be constructed to not expand (e.g., remain a fixed size) during passage of the ingestible electronic device through the gastrointestinal tract of a subject. Again, this may result in the ingestible electronic device being retained within the stomach of a subject for relatively short durations as compared to typical devices that are retained within the stomach of a subject for long durations. Additionally, in some embodiments, an outer dissolvable capsule encapsulating the ingestible electronic device therein may be used, not depicted. In such an embodiment, the outer dissolvable capsule may dissolve once the ingestible electronic device is disposed within the stomach of a subject.

The housing 108 may have any suitable shape. As shown, the housing 108 may be a generally elongated cylindrical shape with hemispherical ends, though any other appropriate shape may also be used. In some embodiments the housing 108 may be cylindrically shaped, spherically shaped, rectangularly shaped, conically shaped, or any other suitable shape. Additionally, embodiments combining two or more shapes are also contemplated. For example, the housing 108 may contain a spherical portion and a cylindrical portion, a cylindrical portion and a conical portion, or any other suitable combination of shapes. Accordingly, it should be understood that the housing of the devices disclosed herein are not limited to any particular shape or configuration.

The housing 108 may be made of any suitable material. For example, in some embodiments, the housing 108 may be made of a biocompatible plastic, such as PVC, polyethylene, polycarbonate, PEEK, Ultem PEI, polypropylene, polysulfone, or polyurethane. Of course, embodiments using other biocompatible materials are also contemplated. Additionally, the electrodes 106 may be made of any suitable material including, for example, platinum, zinc, nickel, and/or any other suitable conductive material that is compatible with the gastrointestinal tract of a subject.

As noted above, the housing 108 may include an exterior surface 112. The two or more electrodes 106 may be disposed on the exterior surface 112 of the housing 108 so that the electrodes may apply electrical stimulation to the tissue of the stomach of the subject as described above. The two or more electrodes 106 may be arranged on the exterior surface 112 of the housing 108 in any suitable pattern. For example, in some embodiments, the electrodes 106 may be arranged in a helical pattern where the two electrodes are wrapped around a circumference of the housing and extend along at least a portion of the length of the housing in the noted helical pattern, see FIG. 1A. This may advantageously permit the electrodes to contact tissue adjacent to the capsule regardless of the specific surface of the cylindrical portion of capsule that is oriented vertically downwards. However, the electrodes 106 may be arranged in other patterns including circular patterns, scattered patterns, linear patterns or any other suitable type of pattern. Additionally, embodiments in which multiple pairs of electrodes are disposed on different portions of a housing to permit electrical contact to be made with tissue adjacent to the different portions of the device are also contemplated.

As shown in FIG. 1B, in some embodiments, an ingestible electronic device 100 may contain internal electronic components, such as a power source 102 and/or a processor 104 with associated non-transitory processor readable medium including processor readable instructions stored thereon. The power source 102 may be a battery, a capacitor, a super capacitor, or any other suitable type of power source. To provide the desired functionality, the processor 104 may be configured to control the operation of one or more components of the ingestible electronic device 100. For example, the processor 104 may be configured to control the power source, an electrical switch electrically connecting the power source and electrodes, and/or any other appropriate component capable of controlling the electrical stimulation applied by the electrodes. For example, the processor 104 may be capable of controlling operation of the device to apply electrical stimulation to the tissue within the stomach of the subject while the ingestible electronic device 100 is disposed within the stomach and ending the electrical stimulation once the ingestible electronic device 100 passes out of the stomach.

In some embodiments, the ingestible electronic device 100 may include one or more sensors 114 that are operatively coupled to the processor 104 as described above. In such embodiments, the sensors may be powered via the power source 102. Further the sensors may be configured to sense or determine one or more operating parameters associated with operation of the device and/or a surrounding external environment. The sensors may then communicate a signal associated with the one or more operating parameters to the processor 104. For example, a sensor may sense a parameter associated with the surrounding external environment, such as pH, that may be used by the processor to determine if the ingestible electronic device 100 is within the stomach of the subject or whether the ingestible electronic device 100 is making contact with the tissue within the stomach of the subject.

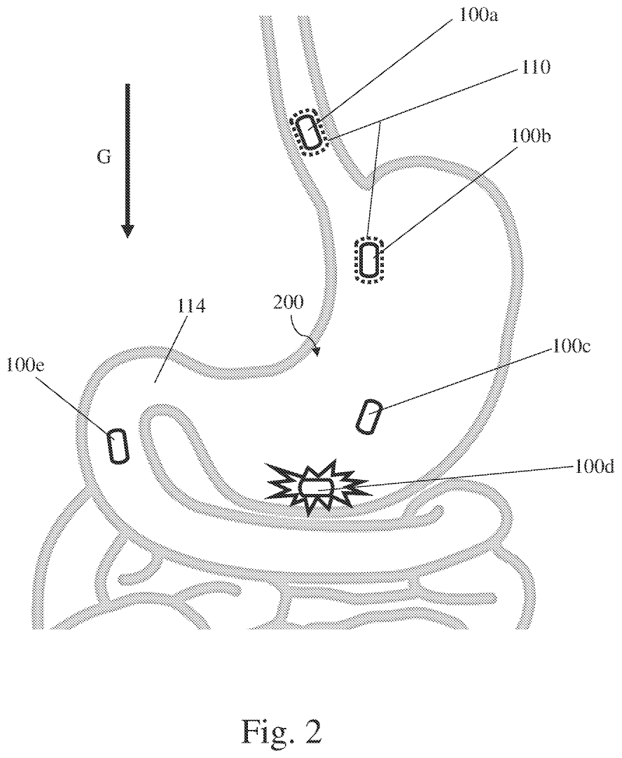

FIG. 2 shows an illustrative embodiment of an ingestible electronic device traveling through a stomach 200 of an exemplary subject. In some embodiments, the ingestible electronic device 100 may be administered to the subject orally such that the ingestible electronic device 100 passes through the esophagus at location 100a and sinks to the bottom of the stomach 200 until the ingestible electronic device 100 contacts the stomach tissue (e.g., ingestible electronic device is at least partly supported by stomach tissue located vertically below the device). In some instances, the ingestible electronic device 100 travels through the stomach under a force of gravity G. Correspondingly, the ingestible electronic device 100 may have a density greater than the fluids located within the stomach 200 of the subject (e.g. a density greater than about 1 g/cc³). Due to the anatomy of the stomach, when a subject ingests the device, the device may come to rest on a portion of the subject's stomach that is with 10 cm, 5 cm, or any other appropriate distance of a pyloric orifice 114 of the subject's stomach.

In some embodiments, the ingestible electronic device 100 may include an outer coating 110. The outer coating 110 may be dissolvable within the stomach (e.g., compare location 100b with location 100c). The outer coating 110 may serve to shield the electrodes 106 and/or a trigger of the device disposed on the housing 108 such that the electrodes 106 may not begin to electrically stimulate tissue until the ingestible electronic device 100 enters the stomach 200. Accordingly, the outer coating 110 may be made of a material that may be dissolved within the stomach 200 of the subject. For example, outer coating 110 may begin to dissolve when the ingestible electronic device comes into contact with the fluids in the stomach 200, see location 100b. As noted above, as the ingestible electronic device continues to descend within the stomach 200 (e.g., under the force of gravity G) at 100c, the ingestible electronic device 100 may travel towards, and come into contact with, a portion of the stomach 200 located vertically below the device at 100d.

It may be desirable to ensure that one of the faces of the device including one or more pairs of electrodes disposed thereon will be in contact with an internal surface of the stomach when the device is resting on a portion of the stomach. For example, a shape of the device's outer housing and a locations and arrangement of the one or more pairs of electrodes may ensure that at least one pair of the electrodes is in contact with the underlying portion of the stomach using any of the previously described arrangements. In either case, once the ingestible electronic device approaches and/or comes into contact with the tissue of stomach 200, for example at location 100d, the ingestible electronic device 100 may apply electrical stimulation to the tissue of the stomach 200 proximate to the device using the associated electrodes, and as described above. Once the ingestible electronic device 100 finishes applying electrical stimulation to the tissue of the stomach 200, the ingestible electronic device 100 may leave the stomach 200 via a pyloric orifice 114 of the stomach and subsequently be excreted from the subject via the subjects GI tract (see, e.g., location 100e) using the natural processes of the gastrointestinal tract and without actively retaining the device in the stomach of the subject. As noted above, the device may stop applying electrical stimulation to the surrounding tissue either prior to, or after detecting, that the device has passed out of the stomach. Again, this may either be done using a predetermined time threshold, a sensed environmental parameter (e.g. pH), or any other appropriate operating parameter.

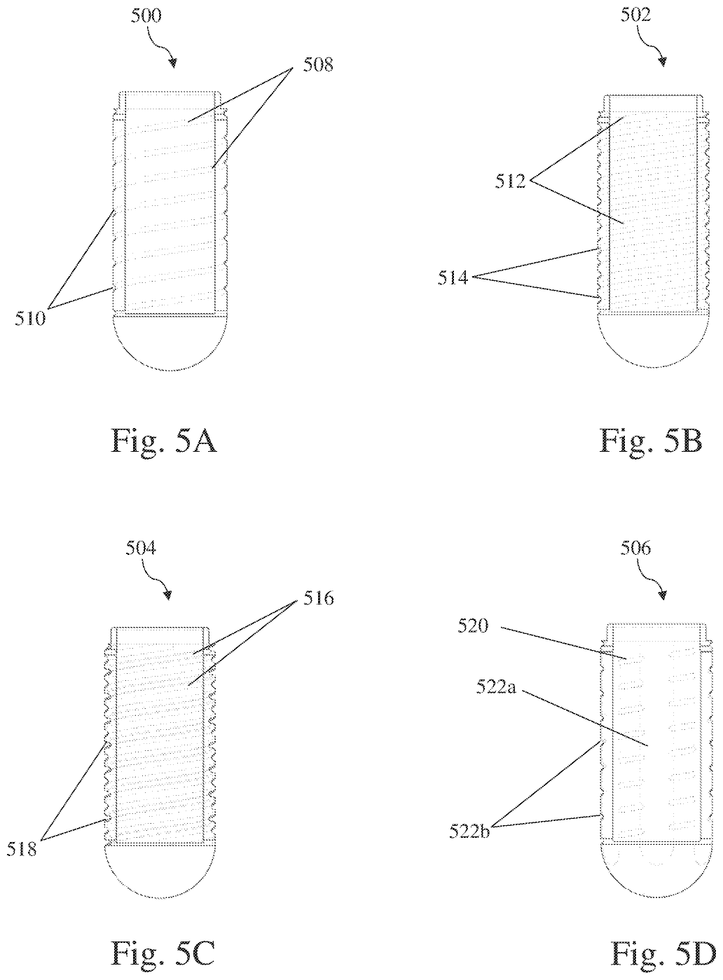

In some instances, one or more gastrointestinal fluids may block or otherwise impede contact between one or more electrodes of the ingestible device and the adjacent tissue (e.g., by increasing an electrical impedance between the one or more electrodes and the tissue). Thus, the Inventors have recognized the advantages of an ingestible device capable of wicking the one or more gastrointestinal fluids from the electrodes. For example, FIGS. 5A-5D illustrate various embodiments of ingestible electronic devices having grooves configured to wick one or more gastrointestinal fluids. In particular, FIG. 5A shows a first embodiment of an ingestible device 500 having an alternating arrangement of electrodes 508 and grooves 510 extending around at least a portion of the body. In this case the one or more electrodes and grooves both spiral around the housing though other arrangements are also contemplated. FIG. 5B shows a second embodiment of an ingestible device 502 having electrodes 512 and grooves 514 extending around at least a portion of the body with a smaller pitch and electrode width as compared to FIG. 5A. FIG. 5C shows a third embodiment of an ingestible device 504 having electrodes 516 and grooves 518 again arranged in an alternating spiral arrangement with deeper channels. FIG. 5D shows a fourth embodiment of an ingestible device 506 having electrodes 520 extending longitudinally along at least a portion of the housing. The depicted embodiment may include primary channels 522a positioned between adjacent electrodes and extending along at least a portion of a length of the device. The device may also include secondary grooves 522a formed in one or more of the electrodes. In the depicted embodiment, the secondary grooves may be arranged in a broken helical pattern spiraling around the electrodes though other arrangements are also contemplated.

As will be appreciated by one of skill in the art, various parameters associated with the electrodes and the grooves may be chosen to adapt the ingestible device to various applications. Such parameters may include groove pitch (e.g., a distance between adjacent portions of the groves), groove width (e.g., a maximum transverse dimension of a groove in a plane parallel to an underlying surface of the ingestible device), groove depth (e.g., a maximum transverse dimension of a groove in a plane perpendicular to an underlying surface of the ingestible device), a shape of the grooves, a layout of the grooves, and/or any other suitable parameter. Of course, other suitable parameters may be modified, depending on the application, as the disclosure is not so limited in this regard.

As discussed herein, the grooves may take on any suitable shape. In some embodiments, the grooves may take on a generally triangular shape, while in other embodiments, the grooves may take on a primarily rectangular or square-like shape. Of course, the grooves may take on any suitable shape, depending on the application, as the disclosure is not so limited in this regard.

Alternatively or in addition, the grooves may take on any suitable layout. For example, in some embodiments, the grooves may be generally helical along an exterior surface of the ingestible device, while in other embodiments, the grooves may be axially arranged. The grooves and electrodes may also extend along a portion, or an entire, length of a device as the disclosure is not limited in this manner. Of course, the grooves may be arranged in other suitable layouts, depending on the application, as the disclosure is not so limited in this regard.

In some embodiments, a pitch of the grooves may be arranged in any suitable manner. For example, in some embodiments, the pitch may be constant along an exterior surface of the ingestible device, while in other embodiments, the pitch may be variable along an exterior surface of the ingestible device. Of course, the grooves may take on any suitable pitch, depending on the application, as the disclosure is not so limited in this regard.

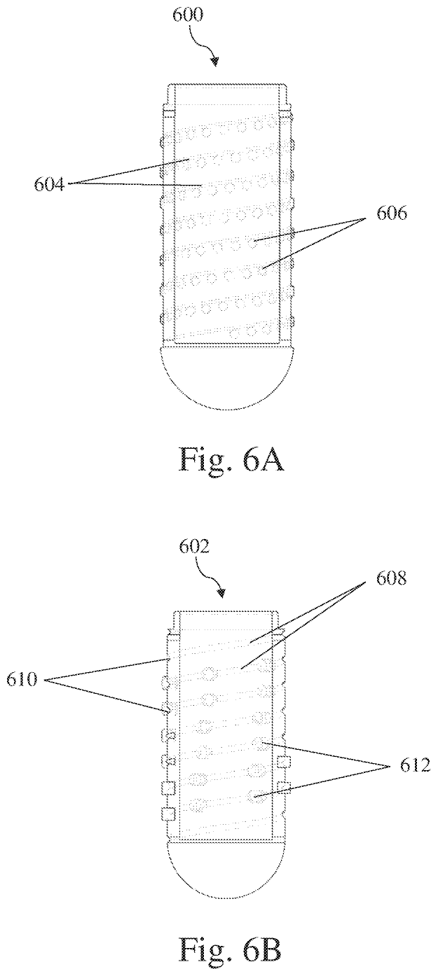

Alternatively or in addition, the ingestible device may include one or more posts, or a plurality of posts, that extend radially outwards from an exterior surface of the ingestible device in order to space at least a portion of the exterior surface of the device from the underlying tissue. Accordingly, the one or more posts may be configured to facilitate wicking of the one or more gastrointestinal fluids from the exterior surface of the ingestible device. For example, FIGS. 6A-6B illustrate two embodiments of an ingestible device including posts configured to form a gap between the exterior surface of the device and the underlying tissue when the device is positioned in the stomach such that the gastrointestinal fluid is wicked away from the portion of the exterior surface of the ingestible device oriented towards the tissue. In the embodiment, of FIG. 6B, an ingestible device 602 includes electrodes 608 along with grooves 610 and posts 612, which are configured to wick fluid away from an exterior surface of the ingestible device (e.g., as described herein).

As will be appreciated by one of skill in the art, various parameters associated with the posts may be configured to adapt the ingestible device to various applications. Such parameters may include post pitch (e.g., a distance between adjacent posts), post width (e.g., a maximum transverse dimension of a post in a plane parallel to an underlying surface of the ingestible device), post height (e.g., a maximum transverse dimension of a post in a plane perpendicular to an underlying surface of the ingestible device), a shape of the posts, a layout of the posts, and/or any other suitable parameter. For example, the posts described herein may have similar widths, heights, and/or pitches as the grooves described herein though other appropriate range of dimensions may also be used. Additionally, posts may exhibit other suitable parameters depending on the application, as the disclosure is not so limited in this regard.

As discussed herein, the posts may take on any suitable shape. In some embodiments, the posts may take on a generally cylindrical shape (e.g., circular, triangular, square, rectangular etc.), mushroom head shape, and other appropriate shapes. Of course, the posts may take on any suitable shape, depending on the application, as the disclosure is not so limited in this regard.

Alternatively or in addition, the grooves may take on any suitable layout. For example, in some embodiments, the posts may be arranged in a generally helical spiral along an exterior surface of the ingestible device, while in other embodiments, the posts may be axially arranged in a plurality of rows along at least a portion of the device. Of course, the posts may be arranged in other suitable layouts, depending on the application, as the disclosure is not so limited in this regard.

As will be appreciated by one of skill in the art, an ingestible device may be equipped with any combination of grooves and/or posts. For example, as shown in FIGS. 5A-5D, in some embodiments, an ingestible device may include only grooves configured to wick one or more gastrointestinal fluids, as described herein. As shown in FIG. 6A, in some embodiments, an ingestible device may include only posts configured to wick one or more gastrointestinal fluids, as described herein. As shown in FIG. 6B, in some embodiments, an ingestible device may include both posts and grooves configured to wick one or more gastrointestinal fluids, as described herein. Of course, the ingestible device may include other features or combinations of features configured to wick one or more gastrointestinal fluids, depending on the application, as the disclosure is not so limited in this regard.

### Example: Experimental Electrical Stimulation Results

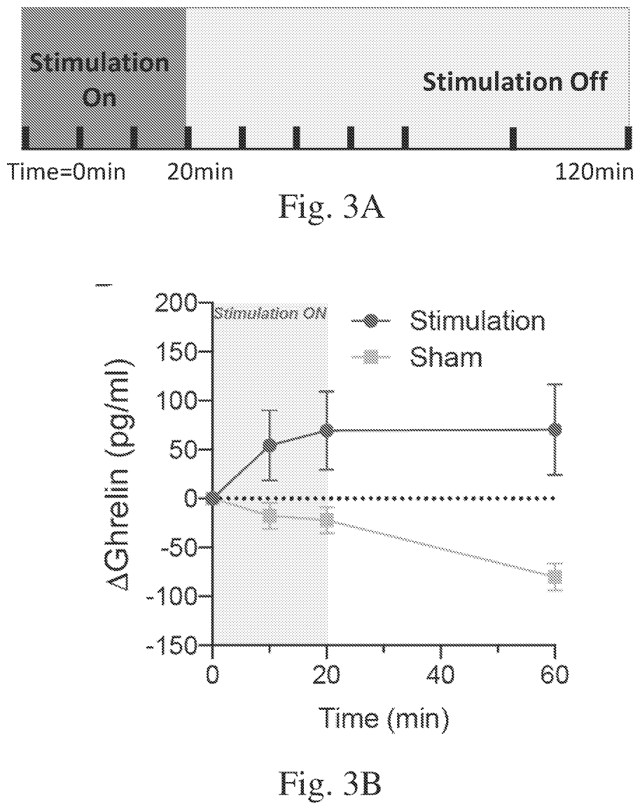

FIGS. 3A-3B show the results of an experiment conducted by the Inventors comparing the Ghrelin levels in an experimental subject to the electrical stimulus exposure time of the stomach tissue of the subject. In the experimental setup, the Inventors applied electrical stimulation pulses to the subject using a 14 Hz pulse frequency rate, a 0.33 ms pulse duration, and a 5 mA current. In the experiment, the electrodes were positioned in a helical pattern on a surface of the device similar to that shown in FIG. 1A. The electrodes were place in contact with the gastric mucosa, approximately 2 cm away from the pyloric orifice. During testing, the Inventors measured Ghrelin levels prior to exposing the stomach of the subject to electrical stimulation for 20 minutes. Ghrelin levels where then measured starting at 5 minutes post stimulation until 120 minutes following the initial electrical stimulation (i.e., 100 minutes after the electrical stimulation was complete). The Inventors observed a substantial increase in Ghrelin levels within the subject when comparing average change in Ghrelin levels at the 5 minute mark and at the 90 minute mark for the stimulation curve versus the sham curve where stimulation was not applied. Specifically, the Inventors observed a change in Ghrelin plasma concentration relative to a baseline Ghrelin plasma level of greater than approximately 100 pg/ml at the 5 minute mark after the initial stimulation and a change in Ghrelin plasma concentration of greater than 200 pg/ml at the 90 minute mark. This confirms that relatively short duration exposure of the stomach tissue located near the pyloric orifice of a stomach of a subject to electrical stimulation may result in clinically relevant changes in the Ghrelin plasma concentration in a subject.

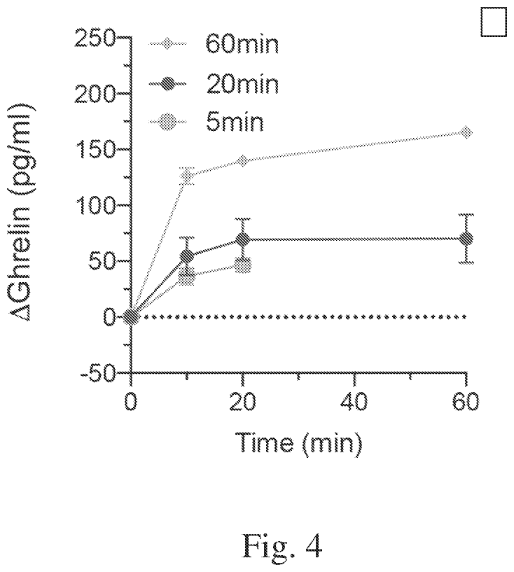

FIG. 4 shows an experiment similar to that described above relative to FIGS. 3A-3B. However, in this experiment, stimulation was applied to a subject's stomach for 5 minutes, 20 minutes, and 60 minutes during separate trials. The resulting changes in Ghrelin plasma concentration versus time for the different durations were compared to a control were no electrical stimulation was applied. As shown in the graph, each stimulation test exhibited significant changes in Ghrelin plasma concentration shortly after the initiation of electrical stimulation regardless of the duration of electrical stimulation. Additionally, the final resulting measured changes in Ghrelin plasma concentrations did not show any apparent distinction based on the length of stimulation applied to the stomach of the subject for the tested stimulation durations between five minutes and 60 minutes.

### Example: Experimental Impedance of Electrical Stimulation within GI Tract

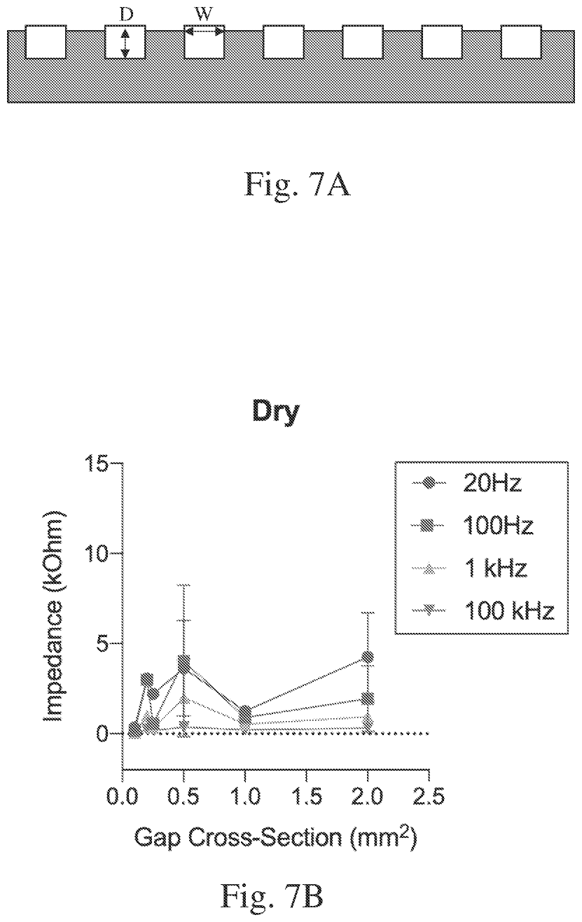

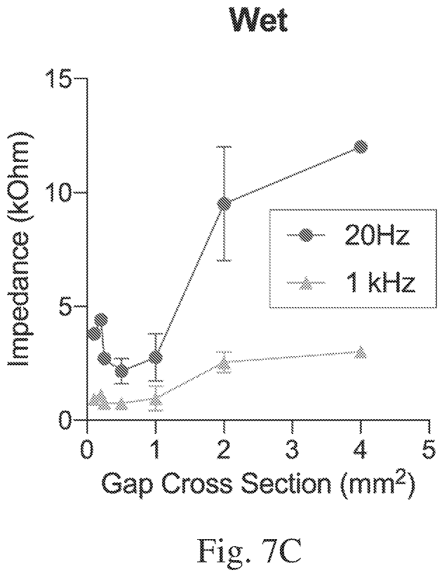

FIGS. 7B-7C show comparative impedance results of an experimental ingestible device in dry and wet conditions respectively. Impedance is shown on the Y-axis, while Gap Cross-Section (e.g., an area of the gap defined by the product of a depth D and a width W, as shown in FIG. 7A). Results were measured with a variety of electrode pulse frequencies. The results of this experiment show that for smaller gap cross-sections, a lower impedance may be achieved.

### Example: Experimental Wicking Configurations

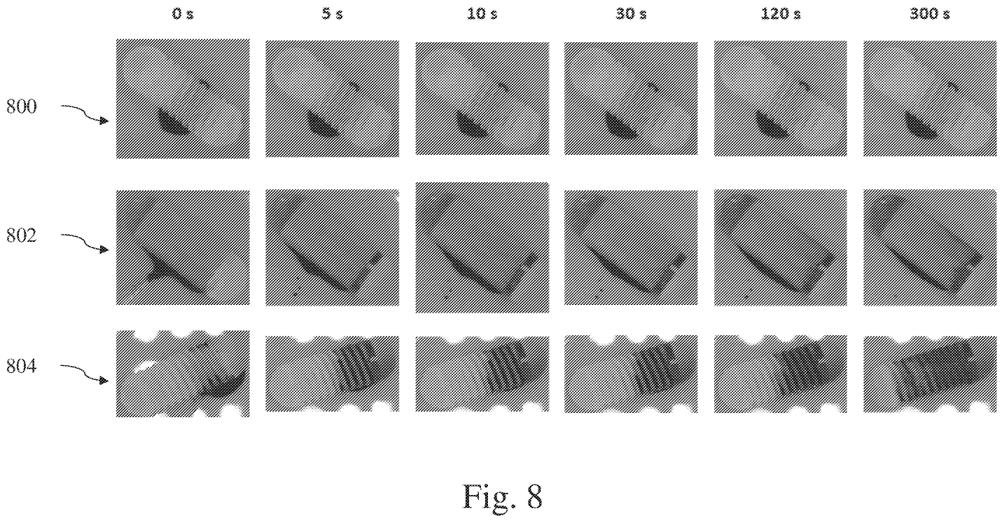

FIG. 8 shows three embodiments of an ingestible device 800, 802, 804 under experimental conditions. In particular, an experiment to measure fluid wicking was performed on the three embodiments of the ingestible device 800, 802, 804. The ingestible device 800 includes a plurality of grooves and no surface treatment. The ingestible device 802 includes no grooves and a surface treatment. The ingestible device 804 includes both grooves and a surface treatment. In the illustrated embodiments, the surface treatment used is an O₂ plasma treatment at 50 W for 5 minutes, though in other embodiments alternative or additional surface treatments may be employed. To measure wicking, the respective ingestible devices 800, 802, 804 were each deposited into a 100 μL methylene blue solution. Snapshots of the respective ingestible devices 800, 802, 804 were taken at consecutive times, as shown in FIG. 8.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided as a single processor or multiple processors. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the embodiments described herein may be embodied as a processor readable storage medium (or multiple computer readable media) (e.g., a computer memory, one or more floppy discs, compact discs (CD), optical discs, digital video disks (DVD), magnetic tapes, flash memories, RAM, ROM, EEPROM, circuit configurations in Field Programmable Gate Arrays or other semiconductor devices, or other tangible computer storage medium) encoded with one or more programs that, when executed on one or more computers or other processors, perform methods that implement the various embodiments discussed above. As is apparent from the foregoing examples, a processor readable storage medium may retain information for a sufficient time to provide processor-executable instructions in a non-transitory form. Such a processor readable storage medium or media can be transportable, such that the program or programs stored thereon can be loaded onto one or more different computing devices or other processors to implement various aspects of the present disclosure as discussed above. As used herein, the term “processor-readable storage medium” encompasses only a non-transitory processor-readable medium that can be considered to be a manufacture (i.e., article of manufacture) or a machine. Alternatively or additionally, the disclosure may be embodied as a processor readable medium other than a computer-readable storage medium, such as a propagating signal.

The terms “program” or “software” are used herein in a generic sense to refer to any type of computer code or set of computer-executable instructions that can be employed to program a computing device or other processor to implement various aspects of the present disclosure as discussed above. Additionally, it should be appreciated that according to one aspect of this embodiment, one or more computer programs that when executed perform methods of the present disclosure need not reside on a single computing device or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. An ingestible electronic device comprising:
a non-expandable housing, wherein the non-expandable housing is configured to pass through a pyloric orifice of the subject without being actively retained in a stomach of the subject; and
at least two electrodes disposed on an exterior surface of the non-expandable housing, wherein the at least two electrodes are configured to apply electrical stimulation to a tissue proximate to the at least two electrodes when the ingestible device is disposed in the stomach of the subject, and wherein the ingestible device is configured to apply the electrical stimulation to the tissue for a time period less than or equal to 1 hour.

2. The ingestible electronic device of claim 1, wherein the time period is greater than or equal to 5 minutes.

3. The ingestible electronic device of claim 1, wherein the ingestible device is configured to pass through the stomach of the subject in less than or equal to 4 hours.

4. The ingestible electronic device of claim 1, wherein the non-expandable housing is at least partially encapsulated in a dissolvable material, wherein the dissolvable material isolates the at least two electrodes from an environment surrounding the ingestible device.

5. The ingestible electronic device of claim 4, wherein the dissolvable material is configured to be dissolved in the stomach of the subject such that once the dissolvable material is dissolved, the two or more electrodes apply electrical stimulation to the tissue proximate to the at least two electrodes.

6. The ingestible electronic device of claim 1, wherein the two or more electrodes extend along a length of the non-expandable housing in a helical pattern.

7. The ingestible electronic device of claim 1, wherein the ingestible device further includes a processor configured to perform the steps of:
determining that the ingestible device is within the stomach of the subject;
applying electrical stimulation to the tissue while the ingestible device is disposed within the stomach; and
ending the electrical stimulation once the ingestible device passes out of the stomach.

8. The ingestible electronic device of claim 1, wherein the ingestible device further includes a trigger configured to activate the two or more electrodes.

9. A method of applying electrical stimulation to tissue within a stomach of a subject, the method comprising:
positioning an ingestible electronic device in the stomach of the subject without actively retaining the ingestible electronic device in the stomach; and
applying electrical stimulation to a tissue of the stomach of the subject proximate to the ingestible electronic device for a time period less than or equal to 1 hour.

10. The method of claim 9, wherein the time period is greater than or equal to 5 minutes.

11. The method of claim 9, further including passing the ingestible electronic device out of the stomach of the subject.

12. The method of claim 11, wherein the ingestible device passes through the stomach in less than or equal to 4 hours.

13. The method of claim 9, further including sensing that the ingestible device is within the stomach of the subject, and wherein applying the electrical stimulation to the tissue of the stomach is based at least partly on sensing that the ingestible device is within the stomach.

14. The method of claim 9, wherein applying the electrical stimulation to the tissue of the stomach includes applying the electrical stimulation using electrodes extending along a length of a housing of the ingestible electronic device in a helical pattern.

15. The method of claim 9, further including dissolving a dissolvable material encapsulating the ingestible device.

16. An ingestible electronic device comprising:
a non-expandable housing, wherein the non-expandable housing is configured to pass through a pyloric orifice of the subject without being actively retained in a stomach of the subject; and
at least two electrodes disposed on an exterior surface of the non-expandable housing, wherein the at least two electrodes are configured to apply electrical stimulation to a tissue proximate to the at least two electrodes when the ingestible device is disposed in a stomach of a subject, wherein the at least two electrodes are configured to contact tissue disposed on multiple sides of the non-expandable housing.

17. The ingestible device of claim 16, wherein the ingestible device is configured to pass through the stomach of the subject in less than or equal to 4 hours.

18. The ingestible device of claim 16, wherein the two or more electrodes extend along a length of the non-expandable housing in a helical pattern.

19. The ingestible device of claim 16, wherein the at least two electrodes include a first pair of electrodes and a second pair of electrodes, wherein the first pair of electrodes is disposed on a first portion of the non-expandable housing and the second pair of electrodes is disposed on a second portion of the non-expandable housing, wherein the first and second portions of the non-extendable housing are located opposite from one another on the housing.

20. The ingestible device of claim 16, wherein the at least two electrodes are configured to apply the electrical stimulation to the tissue for a time period less than or equal to 1 hour.

21. The ingestible device of claim 16, wherein the non-expandable housing is at least partially encapsulated in a dissolvable material, wherein the dissolvable material isolates the at least two electrodes from an environment surrounding the ingestible device.

22. The ingestible device of claim 21, wherein the dissolvable material is configured to be dissolved in the stomach of the subject such that once the dissolvable material is dissolved, the two or more electrodes apply electrical stimulation to the tissue proximate to the at least two electrodes.

23. The ingestible device of claim 1, wherein the exterior surface of the non-expandable housing includes one or more grooves configured to wick one or more gastrointestinal fluids in a region between the exterior surface of the non-expandable housing and the tissue.

24. The ingestible device of claim 1, wherein the exterior surface of the non-expandable housing includes one or more grooves configured to decrease an impedance between the electrodes and the tissue.

25. The ingestible device of claim 1, wherein the exterior surface of the non-expandable housing includes one or more posts configured to wick one or more gastrointestinal fluids in a region between the exterior surface of the non-expandable housing and the tissue.

26. The ingestible device of claim 1, wherein the exterior surface of the non-expandable housing includes one or more posts configured to decrease an impedance between the electrodes and the tissue.
